(19) United States
(12) Patent Application Publication
(10) Pub. No.: US 2004/0063694 A1
(43) Pub. Date: Apr. 1, 2004

(54) Compound useful for the treatment of CNS disorders

(76) Inventor: Monte R. Browder, Plantation, FL (US)

Correspondence Address: IVAX CORPORATION, 4400 Biscayne Boulevard, Miami, FL, 33137, US

(21) Appl. No.: 10/453,082

(22) Filed: Jun. 3, 2003

Related U.S. Application Data
(60) Provisional application No. 60/386,126, filed on Jun. 5, 2002.

(51) Int. Cl. A61K 31/551; C07D 513/14

(52) U.S. Cl. 514/220; 540/560

(57) ABSTRACT

The present invention relates to the compound 2-ethyl-10-(4-methyl-1-piperazinyl)-4H-thieno[2,3-b][l,5]benzodiazepine and its use as is or in substantially pure form in the treatment of CNS disorders including schizophrenia and bipolar disorder. The compound is prepared by reacting a substituted thiophene compound with 2-fluoronitrobenzene to form, after ring closure and reaction with N-methylpiperazine, the title compound. The compound is then purified to substantially pure form and is useful as an antipsychotic. The compound may also be combined with other active ingredients including HMG CoA reductase inhibitors such as lovastatin or simvastatin and/or antidepressants such as fluxoetine or other SSRIs to form medically useful combination products useful in treating psychotic conditions and depression while also preventing any rise beyond the normal range of cholesterol levels in any subset of patients that might develop such a condition.

### BACKGROUND OF THE INVENTION

The present invention relates to substantially pure forms and physical forms thereof of 2-ethyl-10-(4-methyl-1-piperazinyl)-4H-thieno[2,3-b][1,5]benzodiazepine. In addition, the invention relates to the uses of this compound and forms thereof to treat central nervous system disorders including schizophrenia. The compound in impure form was originally disclosed in U.S. Pat. No. 4,115,574. Following the grant of this patent and subsequent development of a similar drug (e.g. olanzapine) which was specifically claimed in U.S. Pat. No. 5,229,382, the present inventors have discovered that, in fact, the 2-ethyl-10-(4-methyl-1-piperazinyl)-4H-thieno[2,3-b][1,5]benzodiazepine is useful in its own right as a central nervous system drug. In particular, the inventors have discovered that, contrary to the data presented in the '382 patent with respect to the purported cholesterol raising effects of this compound in dogs and this correlation to humans, the compound in substantially pure form as well as its use in any form in pharmaceutical compositions in treating CNS disorders in human beings, does not significantly effect or elevate cholesterol in humans and the differences relative to olanzapine are, in fact, not statistically significant.

The 2-ethyl compound of the present invention has pre-clinical in vitro and in vivo data as presented in Chakabarti et al. in J. Med. Chem. 1980, 23, pp 878-994. Therein, the data presented for compound 6 (the 2-ethyl compound of the present invention) shows that rat conditioned avoidance test data (2 (31-50% block)(10 mg/kg)) was higher than the data obtained for the rat catalepsy test (1(16 mg/kg)) indicating that the compound is at least a potential development candidate because of the lower dose needed for activity versus the higher dose that might predict catalepsy/diskenisia (extra pyramidal side effect) potential. However, as stated in the '382 patent and other related Lilly patents to olanzapine, the ethyl compound allegedly had some cholesterol raising effect in beagle dogs and was thus purportedly not a viable development candidate. The present inventors suggest otherwise. There is a need for alternative new chemical entities that treat CNS disorders in human beings.

### SUMMARY OF THE INVENTION

The present invention comprises 2-ethyl-10-(4-methyl-1-piperazinyl)-4H-thieno[2,3-b][1,5]benzodiazepine, or an acid addition salt thereof or prodrug thereof in substantially pure form. The compound may be in the form a substantially pure form of crystalline material or wherein the substantially pure form is an amorphous material. In addition, the compound is a crystalline hydrate or hemi-hydrate or is in the form of a polymorph. Each of these substantially pure forms or any form of the 2-ethyl compound may be formulated into and comprise a pharmaceutical composition comprising the 2-ethyl compound or a pharmaceutically acceptable acid addition salt or prodrug thereof and a pharmaceutically acceptable excipient. The pharmaceutical composition may be in the form of a pharmaceutical composition in capsule, tablet or liquid form comprising from about 0.5 mg to about 20 mg of the ethyl compound together with a pharmaceutically acceptable excipient or diluent.

The present invention also comprises a method of treating a patient in need of treatment thereof suffering from or susceptible to psychosis, acute mania or mild anxiety states, which comprises administering a pharmaceutically effective amount of 2-ethyl-10-(4-methyl-1-piperazinyl)-4H-thieno[2,3-b][1,5]benzodiazepine, or a pharmaceutically acceptable acid addition salt or prodrug thereof to said patient. It further comprises a method of treating said patient wherein the effective amount is from 0.1 to 20 mg per day of 2-ethyl-10-(4-methyl-1-piperazinyl)-4H-thieno[2,3-b][1,5]benzodiazepine, or a pharmaceutically acceptable acid addition salt or prodrug thereof. The invention also comprises a method of treating a patient suffering from or susceptible to schizophrenia comprising administering to said patient a pharmaceutically effective amount of 2-ethyl-I 0-(4-methyl-1-piperazinyl)-4H-thieno[2,3-b][11,5]benzodiazepine or a salt, prodrug or physical form thereof. The invention also relates to a method of treating said schizophrenic pateient wherein the effective amount is from 0.1 to 20 mg per day of 2-ethyl-10-(4-methyl-1-piperazinyl)-4H-thieno[2,3-b][1,5]benzodiazepine, or a pharmaceutically acceptable acid addition solution salt or prodrug or physical form thereof.

The invention also comprises a pharmaceutical composition comprising a combination of a pharmaceutically effective amount of 2-methyl-10-(4-methyl-1-piperazinyl)-4H-thieno[2,3-b][1,5]benzodiazepine or fluoxetine and 2-ethyl-10-(4-methyl-1-piperazinyl)-4H-thieno[2,3-b][1,5]benzodiazepine, together with pharmaceutically acceptable excipients.

In addition, the invention comprises a method of treating a patient having or susceptible acute mania comprising administering a pharmaceutically effective amount of 2-ethyl-10-(4-methyl-1-piperazinyl)-4H-thieno[2,3-b][1,5]benzodiazepine or a salt or prodrug thereof and a method of treating a patient having mild anxiety states comprising administering a pharmaceutically effective amount of 2-ethyl-10-(4-methyl-1-piperazinyl)-4H-thieno[2,3-b][1,5]benzodiazepine or a salt or prodrug thereof. The invention also comprises a method of treating a CNS disorder or condition comprising administering a pharmaceutically effective amount of the compound 2-ethyl-10-(4-methyl-1-piperazinyl)-4H-thieno[2,3-b][1,5]benzodiazepine or a salt or prodrug or physical form thereof wherein, relative to the compound 2-methyl-10-(4-methyl-1-piperazinyl)-4H-thieno[2,3-b][1,5]benzodiazepine, there is no statistically significant elevation in cholesterol levels in patients treated with said compound.

The present invention also comprises a pharmaceutical composition in liquid form suitable for oral or intravenous administration wherein the composition comprises a compound selected from 2-ethyl-10-(4-methyl-1-piperazinyl)-4H-thieno[2,3-b][1,5]benzodiazepine or salt or prodrug thereof and a pharmaceutically acceptable diluent. In addition, the present invention relates to a sustained release delivery system comprising a compound selected from 2-ethyl- or 2-methyl-10-(4-methyl-1-piperazinyl)-4H-thieno[2,3-b][1,5]benzodiazepine and sustained release excipients or an immediate release delivery system comprising a compound selected from 2-ethyl-10-(4-methyl-1-piperazinyl)-4H-thieno[2,3-b][1,5]benzodiazepine or salt or prodrug thereof and immediate release excipients.

### DETAILED DESCRIPTION OF THE INVENTION

As delineated above, the present inventors have found that the compound 2-ethyl-10-(4-methyl-1-piperazinyl)-4H-thieno[2,3-b][1,5]benzodiazepine or salt or prodrug or physical form thereof including in substantially pure form is a useful compound in its own right to treat CNS disorders and conditions in patients in need of treatment thereof despite suggestions otherwise in the '382 patent and related applications/patents. The file history of the '382 patent also provides numerous toxicity and biological tests which described the overall in vitro and in vivo characteristics of the ethyl compound of the present invention as being similar to olanzapine except for the alleged difference in cholesterol effects. This data per se is incorporated by reference except for the cholesterol data therein which is statistically incorrect and not probative of cholesterol raising effects in human patients.

The 2 ethyl analog of olanzapine (the compound of formula I or the 222 compound) may be prepared synthetically by a process that comprises reacting N-methyl-piperazine with a compound of formula II (Scheme I) wherein Y is a leaving group that is displaced by the NH group of the N-methylpiperidine to form the compound of formula I (2-ethyl compound). Alternatively, the compound of formula I may be prepared by ring closing a compound of formula III (Scheme I). In Scheme I, the Y group may be selected from amino groups or substituted or disubstituted versions thereof (e.g. mono or di C1-C4 alkyl) or hydroxyl, thiol, alkoxy, C1-C4alkylthio, or C1-C4alkylsulphonyl or halogen atom. When Y is, for example, NH2, the compound of formula II may be prepared by condensing a thiophene compound of formula IV (see scheme 1) with an orthohalonitrobenzene in the presence of a base, for example sodium hydride, in a suitable solvent such as THF (tetrahydrofuran) to form a nitronitrile of formula V which can be reduced and ring closed to form the amidine of formula II (see Scheme I). Various other alternative syntheses may be performed according to the processes described in the '382 patent but with replacing the methyl moiety of olanzapine with an ethyl group. The preferred synthetic route to make the ethyl compound of formula I involved reacting a compound of formula IV with 2-fluoronitrobenzene to form the compound of formula V which was reduced and ring closed to form the amidine of formula II. This amidine was then reacted with N-methylpiperazine to form the compound of formula I. The starting materials may be obtained from commercially available sources or, in the case of the substituted thiophene, may be prepared according to the procedure described in Example 1 of the '382 patent using the four carbon analog of propionaldehyde as one of the three reactants to form 2-amino-5-ethylthiophene-3-carbonitrile (formula IV). As additionally disclosed in U.S. Pat. No. 4,115,574, the compound used to treat CNS disorders of this invention may be prepared from 9,10-dihydro-4H-thieno[3,4-b][1,5]benzodiazepine10-one by reacting this intermediate with N-methylpiperidine. In turn, this intermediate may be prepared by oxidizing 9,10-Dihydro-4H-2,5-dihydrothieno[3,4-b][1,5]benzodiazepine-10-one or by hydrolyzing 10-amino-4H-thieno[3,4-b][1,5]benzodiazepine. This 10 amino intermediate may be prepared from intramolecular ring closure of the amino acid intermediate of formula VI (see scheme II). The present invention is also directed to products produced by the process(es) described above. Thus, the present invention is directed to a compound of formula I that is prepared from a compound of formula II or a compound of formula III.

Purification of the compound of formula I to form substantially pure compound that may be in the form of a crystalline anhydrate or in the form of a hemihydrate or hydrate (1-3 mono, di or trihydrate) is achieved by initially purifying the obtained material by chromatographic means (HPLC or column chromatography). Upon more than one crystallization of this material, substantially pure material is obtained that may then be further purified (recrystalized) to form, under anhydrous conditions, crystalline anhydrate in substantially pure form. Similarly, the material obtained from chromatographic means may be recrystallized under the appropriate conditions with the proper molarity of water to form hemihydrate crystalline material or the mono, di or trihydrated material. In addition, and depending upon the particular formulation or dosage form desired, the purified material may be dissolved and blended with a suitable binder such as PVP to form amorphous material that is in substantially pure form.

In order to prepare pure polymorphs of the claimed compound, the compound obtained, for example, as described in U.S. Pat. No. 4,115,574 (“the '574 patent”) or herein in initial form may be dissolved in ethyl acetate under anhydrous conditions (less than 1% water present) to form substantially pure crystalline anhydrate polymorph. The term “substantially pure” means that the particular product has been purified beyond the purification procedures described, for example, in the '574 patent for the preparation of 2-ethyl-10-(4-methyl-1-piperazinyl)-4H-thieno[2,3-b][1,5]benzodiazepine. Therein a yellow solid was obtained, recrystallized one time from hexane (mp 195-197 C) and converted to a maleate salt of unspecified purity (mp 186-188 C). Compound characterization methods including X-ray diffraction studies, differential scanning calorimetry, thermogravimetric analysis, water titration, HPLC, FT-IR and NMR are utilized to differentiate between different polymorphs or physical forms. The melting point of the material obtained via Example 1 below was 203-206 C. The present invention is therefore directed to a compound selected from 2-ethyl-10-(4-methyl-1-piperazinyl)-4H-thieno[2,3-b][1,5]benzodiazepine having a melting point of 203-206 C. The present invention is also directed to a compound having a particular X-ray pattern having d values and relative intensities of 6.56 (13.9%); 6.21 (39.5%); 5.31 (11.7%); 5.01 (29%); 4.71 (15.2%); 4.42 (38.7%); 4.15 (100%); 3.28 (18%) and 3.11 (10.3%).

Prodrugs of this compound or substantially pure versions thereof are also included within the scope of the invention. Prodrugs are defined as any derivative of the compound of formula I that is upon administration to a patient in need of treatment thereof of such prodrug metabolized or otherwise transformed into the compound of formula I. The term “pro-drug” also means compounds that are rapidly transformed in vivo to yield the parent compound of the above formula, for example by hydrolysis in blood. A thorough discussion is provided in T. Higuchi and V. Stella, “Pro-drugs as Novel Delivery Systems”, Vol. 14 of the A.C.S. Symposium Series, and in Bioreversible Carriers in Drug Design, ed. Edward B. Roche, American Pharmaceutical Association and Pergamon Press, 1987.

Pharmaceutically acceptable salts are also included within the scope of the invention and include acid addition salts (e.g. HX wherein X is an anion such as chlorine). Suitable acid addition reactants to form the pharmaceutically acceptable salts include hydrochloric acid, nitric, sulfuric acid or phosphoric acids or organic acids such as glycollic, maleic, hydroxy maleic, fumaric, malic, tartaric, citric or lactic acid or organic sulphonic acids etc. These acid addition salts are also present in many forms including crystalline forms, amorphous forms and as crystalline hydrates or hemihydrates. Formation of the maleic acid salt in impure form is disclosed in the '574 patent (example 26 therein). The present invention excludes as a compound per se the crude compound maleic acid salt disclosed in this reference except for substantially pure versions thereof. Its use, however, is claimed in the treatment of CNS disorders or conditions because of the purported cholesterol raising effect of this compound which the inventors believe is not, in fact, the case.

The active ingredient of the invention may additionally be milled or otherwise transformed to form compositions having particle sizes of between 1 micrometer and 220 micrometer before blending to form a tablet or capsule formulation. Particle size determinations may readily be made by any standard method including, for example, the Malvern method. The present invention therefore relates to a method of treating a patient with a CNS disorder or condition selected from a psychotic condition wherein said patient is treated with a pharmaceutically effective amount of 2-ethyl-10-(4-methyl-1-piperazinyl)-4H-thieno[2,3-b][1,5]benzodiazepine having a particle size before tablet or capsule formation of between 1 to 220 micrometers.

It is known that rate of dissolution of drug particles can be increased by increasing the surface area of the solid, i.e., decreasing the particle size. Consequently, methods of making finely divided drugs have been studied and efforts have been made to control the size and size range of drug particles in pharmaceutical compositions. For example, dry milling techniques have been used to reduce particle size and thereby influence drug absorption. However, in conventional dry milling, as discussed by Lachman et al., The Theory and Practice of Industrial Pharmacy, Chapter 2, “Milling”, p. 45 (1986), the limit of fineness is reached in the region of about 100 .mu.m (=100,000 nm), where the milled material begins to cake onto the surfaces of the milling chamber. Lachman et al. note that wet grinding is beneficial in further reducing particle size, but that flocculation restricts the lower particle size limit to approximately 10 .mu.m (=10,000 nm). Commercial airjet milling techniques have provided particles ranging in average particle size from as low as about 1 .mu.m to 50 .mu.m (=1,000 nm to 50,000 nm). It is not known, however, that the 2-ethyl homolog of this invention is advantageously milled according to the above processes to make the compound having an average particle size of between about 1-200 micrometers. The preferred average particle size is less than 200 micrometers. The 2-ethyl homolog of this invention is slightly less soluble than olanzapine in aqueous systems and thus there is a need for a formulation having the 2-ethyl homolog with reduced particle sizes or below 200 micrometers for enhanced solubility and delivery.

The compound of the invention in all its forms is useful in the treatment of various central nervous system (CNS) disorders or conditions. The compound has been tested using standard behavioral models predictive of antipsychotic activity. These tests include the tests conducted in published papers mentioned above including the rat conditioned avoidance test, the rat catalepsy test and other tests including binding assays to central nervous system receptors including D-1 and D-2 dopamine receptors. In addition, the compound shows activity at the 5HT-2 and 5HT-1 receptors and is useful in treating schizophrenia as well as mania and is also useful in combination therapy with antidespressants such as fluoxetine and other SSRIs and antidepressants (not limited to SSRIs) to treat bipolar disorders.

The substantially pure compounds of the invention and the uses of the compound per se in all its forms are effective over a wide dosage range with the actual dose chosen based upon the route of administration (oral, parentarel, iv etc. but the doses in adult patients will typically run between 0.01 to 40 mgs/day. A once a day dose should be sufficient but may be divided depending upon patient need and provided the maximum recommended daily dose is not exceeded. The dose provided may also be dependent upon the disease state or condition that is being treated with less strength needed for mild anxiety states versus severe schizophrenia. Of course, if necessary the dosing regimen may be titrated to provide the maximum benefit to the patient over time.

The route of administration will also guide the skilled artisan to use either an oral composition or a liquid composition. The present invention relates to pharmaceutical compositions comprising the ethyl-compound as described above in all its alternative forms and a pharmaceutically acceptable excipient or diluent. The most preferred dosage form is an oral dosage form as an immediate release tablet or capsule. Tablets may be prepared by conventional means using dry blend or wet granulation technology to mix the active ingredients and the immediate release excipients including binders, lubricants, polymers etc., fillers and other conventional immediate release excipients well known in the art to form a tablet that provides an immediate release of the active ingredient into the patient upon administration and ingestion of the tablet form.

In some cases, it may be desired to provide a controlled release dosage form that releases the active ingredient in a controlled manner over a twenty-four hour time period. In this case, the pharmaceutical composition comprises a formulation comprising the active ingredient selected from the ethyl analog of olanzapine or physical form thereof and extended release excipients. The extended release excipients may be selected from those conventional extended release dosage forms known in the art to control the release of a drug into the patient including, for example, matrix dosage forms that are erodible over time having those polymers that form matrices in tablet dosage forms or the dosage forms may be of the push-pull variety originally developed by Alza (e.g. osmostic dosage form as disclosed in, for example, 4,327,725) or variants thereof including, for example, the SCOT systems of Andrx (for example, the system described in 5,654,005).

In addition to having the single active ingredient selected from the ethyl homolog of olanzapine, the pharmaceutical compositions of the invention may also further include additional active ingredients selected from, for example, olanzapine (dual antipsychotic formulation) or other active ingredients selected from the antidepressants (e.g. fluoxetine or other SSRIs). These compositions may be formulated by simply adding the additional active ingredient in its known dosage range to provide dual active ingredient compositions and tablets or capsules (immediate or extended release versions). In some combinations, it may be preferable to provide an immediate release form of one active ingredient and an extended or controlled release form of the other active ingredient(s) in the same tablet form. In this case, the active ingredients could be separated into distinct layers (e.g. bi-layer or multi-layer tablet) with the immediate release components being on the outer surface of the dosage form while the controlled release components can be both in the outer layers and in the inner matrix portion of the dosage form depending upon the nature of the targeted release profile. One of ordinary skill in the art can formulate using in vitro dissolution profiles to simulate the IR/CR profile desired to provide to the patient in need of treatment thereof. The in-vivo pharmakokinetic profile of any type of dosage form may be measured by conducting pharmakokinetic studies in normal volunteers.

In addition to sustained release oral dosage forms, the present invention also includes pharmaceutical compositions comprising liquid formulations that are capable of being administered in a sustained release fashion (e.g. injectable sustained release formulations). In this case, the composition comprises the active ingredient (the ethyl compound and suitable forms thereof) along with a sustained release excipient and the liquid vehicle (e.g. waters, mild isotonic salt solutions, pH neutral (6-7) to acidic (pH 5-6)). Intramuscular formulations may be prepared by using suitable oils such as sesame oil along with the active ingredient(s). Starch formulations may also be prepared by combining the active ingredient(s) with starch.

In order to provide a more statistically robust study of the potential cholesterol raising effect in beagle dogs of the ethyl homolog of olanzapine at elevated doses well beyond what a human equivalent dose of the ethyl homolog would be, the following studies are conducted. 45 female beagle dogs are divided into three groups of 15 and randomly grouped together so that each group has roughly similar average weights per dog and similar average total cholesterol levels per dog before administration of the ethyl compound of the invention, olanzapine and a placebo. The dogs are studied over a course of 8 mgs/kg/day per drug for a twenty-six week period and assessed at varying time points for LDL, HDL and total cholesterol as well as having their estrous cycle monitored and measured. The results provide the effect of raising total cholesterol in female beagle dogs attributable to drug versus that attributable to the estrous cycle or variations thereof in the same dogs. Instead of obtaining a total average value of greater than 240 mg/Dl of cholesterol in female beagle dogs, the inventors have discovered that, in fact, the total cholesterol values as a result of a twenty six week administration of the ethyl compound of this invention resulted in total average cholesterol values of 240 or less provided the effect associated with normal variations in estrous cycle is discounted. Because of the hugely elevated doses of the ethyl compound provided to the beagle dogs relative to the normal range of a drug of this kind (e.g. olanzapine at 5-30 mgs/day per person of greater than 70 kgs), any rise in cholesterol in beagle dogs at 8 mg/kg/day cannot be correlated to humans who take far less of an average dose per day of the drug. The preferred normal range in human patients of cholesterol level (total) is described in the Merck Manual as 120-220 mg/Dl. The preferred range, therefore, for the treatment of human patients having a CNS disorder or condition without impacting cholesterol levels comprises treatment of said patients with the 2-ethyl homolog of olanzapine wherein said patients upon at least six weeks of therapy have cholesterol levels of between 120-220 mg/Dl.

In the event olanzapine or the 2-ethyl homolog thereof do raise cholesterol in a subset of human patients taking said medication, the present inventors have also discovered a combination therapy that is effective in preventing or mitigating any rise in cholesterol associated with administration of an antipsychotic such as olanzapine or the 2-ethyl homolog thereof by coadministering an effective dose of an HMG-CoA reductase inhibitor either in the form of separate dosage forms or together in a single dosage form. The preferred HMG CoA reductase inhibitors are those selected from, for example, commercially available drugs such as lovastatin, simvastatin or pravastatin although newer medications such as LIPITOR® or other known commercially available or pending or approved drug applications for lowering cholesterol may also be utilized in the combination treatment. The effective dosage range for either a single tablet or co-administration of separate dosage forms is selected from the minimal approved dose for the HMG CoA reductase inhibitor as delineated on the package insert or in the Physicians Desk Reference for the approved HMG CoA reductase inhibitor. In effect, while certain drugs are considered safe and effective, in some percentage of patients side effects can occur which may require mitigation or discontinuation of the particular drug regimen. In this instance, if olanzapine or the 2-ethyl homolog thereof produces in a subset of patients some elevation in cholesterol levels, it may be beneficial to provide this subset of patients a therapeutically useful alternative that eliminates the side effect and doesn't negatively affect the main therapeutic purpose of the drug-e.g., treatment of a psychotic condition. To that end, it might be useful and beneficial to monitor patients cholesterol levels to suggest individualized treatment regimes that benefit that particular patient. The drugs can be administered in the same dosage form or concomitantly in commercially available dosage forms of the combination regimen. In addition, for that subset of psychotic patients that have elevated cholesterol levels from diet, lack of exercise or genetic predisposition, this subset of patients taking medication such as olanzapine or, upon regulatory approval, the ethyl homolog thereof, these patients would also benefit from such a combination product.

### EXAMPLES

### Example 1

### 2-ethyl-10-(4-methyl-1-piperazinyl)-4H-thieno[2,3-b][1,5]benzodiazepine

The title compound was prepared by reacting 2-amino-3-cyano-5-ethylthiophene with 2-fluoronitrobenzene in a suitable solvent to form the nitronitrile as shown in Scheme IV. The nitronitrile was then reduced and ring closed under suitable conditions to form the amidine of formula II. This amidine was then reacted with N-methylpiperazine to form the title compound that was subsequently purified and repurified to substantially pure form. NMR data confirmed the structure of the compound. The melting point of the title compound upon purification was 203-206 C.

X-ray powder diffraction analysis was performed on the above sample which was analyzed on a Siemens D55 Automated Powder Diffractometer (Insturment ID No. LD-301-4) which was operated according to the standard operating procedure EQ-27, Rev. 12. The Diffractometer was equipped with a graphite monochromator and a Cu (lamda=1.54 Angtroms) X-ray source operated at 50 kV, 40 mA. Two-theta calibration was performed using an NBS mica standard (SRM675). The sample was analyzed using the following instrument parameters: measuring range 4-40° 20; Step width 0.050° and measuring time per Step 1.2 seconds. Sample preparation was performed using a front pack sample plate. The results were presented as two theta diffraction angles, d-spacings and intensities as shown below:

| d value | 2-Theta | Intensity | Intensity % |
|---|---|---|---|
| 9.95776 | 8.873 | 290 | 1.8 |
| 7.82679 | 11.296 | 904 | 5.7 |
| 7.24121 | 12.213 | 1340 | 8.5 |
| 6.69772 | 13.208 | 1247 | 7.9 |
| 6.56591 | 13.474 | 2203 | 13.9 |
| 6.21278 | 14.244 | 6250 | 39.5 |
| 5.69868 | 15.537 | 775 | 4.9 |
| 5.31647 | 16.661 | 1846 | 11.7 |
| 5.01460 | 17.672 | 4591 | 29.0 |
| 4.71434 | 18.808 | 2407 | 15.2 |
| 4.42359 | 20.056 | 6128 | 38.7 |
| 4.14954 | 21.396 | 15833 | 100.0 |
| 4.01214 | 22.138 | 2149 | 13.6 |
| 3.94472 | 22.521 | 1321 | 8.3 |
| 3.83051 | 23.202 | 995 | 6.3 |
| 3.75906 | 23.649 | 2209 | 14.0 |
| 3.59146 | 24.769 | 1547 | 9.8 |
| 3.52979 | 25.209 | 365 | 2.3 |
| 3.39238 | 26.248 | 878 | 5.5 |
| 3.28141 | 27.153 | 2857 | 18.0 |
| 3.25209 | 27.402 | 2326 | 14.7 |
| 3.15521 | 28.261 | 887 | 5.6 |
| 3.11034 | 28.677 | 1635 | 10.3 |
| 3.07268 | 29.036 | 555 | 3.5 |
| 3.03293 | 29.425 | 379 | 2.4 |
| 2.97364 | 30.026 | 280 | 1.8 |
| 2.93099 | 30.483 | 419 | 2.6 |
| 2.88982 | 30.918 | 1537 | 9.7 |
| 2.82057 | 31.697 | 465 | 2.9 |
| 2.75335 | 32.492 | 407 | 2.6 |
| 2.72065 | 32.893 | 695 | 4.4 |
| 2.64722 | 33.833 | 556 | 3.5 |
| 2.58888 | 34.619 | 366 | 2.3 |
| 2.56517 | 34.949 | 443 | 2.8 |
| 2.53469 | 35.383 | 421 | 2.7 |
| 2.47433 | 36.276 | 706 | 4.5 |
| 2.38186 | 37.737 | 621 | 3.9 |
| 2.36277 | 38.053 | 434 | 2.7 |
| 2.30329 | 39.075 | 247 | 1.6 |

The present invention comprises a compound including but not limited to a substantially pure compound having the X-ray diffraction pattern as shown above. The present invention comprises a compound having the X-ray pattern shown above having the peak values with the relative intensities of greater than 20%. It further comprises pharmaceutical compositions having a substantially pure compound with said X-ray pattern and to methods of treating central nervous system diseases with a compound having such an X-ray pattern or sub-pattern with the 20% or greater peak relative intensities.

### EXAMPLE 2

Tablet Formulations:

The following doses per tablet of the ethyl compound of example 1 are provided: 2.5 mg, 5 mg, 7.5 mg, 10 mgs or 15 mgs (QD). For an immediate release tablet formulation, the following inactive ingredients may also be added: carnauba wax or suitable equivalent; crospovidone, HPC, HPMC, (binders) lactose (fillers), magnesium stearate (lubricant), microcrystalline cellulose (disintegrant) and conventional dyes.

A Zydis formulation may be prepared using the above active ingredient doses and orally disintegrating excipients selected from, for example, gelatin, mannitol, aspartame, sodium methyl paraben and sodium propyl paraben.

Each dosage form is prepared using standard formulation techniques for the immediate release conventional or Zydis formulation according to procedures well known in the art.

### EXAMPLE 3

Clinical trials of patients having manifestations of a psychotic disorder such as schizophrenia or bipolar mania are conducted over a six week period using an oral dosage form of the claimed drug at different dosages as indicated above. The pre-clinical data demonstrate utility of the claimed invention in the treatment of CNS disorders or conditions.

### EXAMPLE 4

Combination tablets or dosage forms may be prepared according to conventional tableting or dosage form preparation methods to form a tablet having two or more active ingredients selected from olanzapine or the 2-ethyl homolog thereof and an additional active ingredient selected from an antidepressant (SSRIs such as fluoxetine) and/or an HMG CoA reductase inhbitor along with conventional immediate release, quick dissolving or extended release formulation excipients depending upon the type of dosage form and the release profile desired for the particular combination of drugs to treat or prophylactically prevent a condition or disease or potential elevation in cholesterol levels that might occur in a subset of patients treated with olanzapine or the 2-ethyl homolog thereof.

### EXAMPLE 5

A dog study was conducted by orally administering a high dose of the 2,2,2-compound (the 2-ethyl homolog of olanzapine) (8 mgs/kg/day); a placebo and olanzapine (8 mgs/kg/day) over a twenty-six week period. Cholesterol, progesterone and multiple other parameters were measured over the course of the study. Fifteen animals (female beagle dogs) were used in each arm of the study. The mean total cholesterol values after the full duration of the study was 186.8 mgs/dL (222); 155.5 (control); and 143.7 (olanzapine). Each of these average values at the end of the study and for most of the duration of the study was well within the normal healthy reference range for female beagle dogs. The 222 average value spiked to 237.2 mgs/dL at a single point early on in the study but rapidly went down over the remaining part of the study to the 186.8 average value. Individual animals in the 222 group appeared to have cholesterol values that were correlated with estrous and a rise in progesterone levels. The peak in the progesterone level appeared approximately one month before to right before the peak in cholesterol values and suggested that the rise in cholesterol values in individual animals was due to the rise in progesterone associated with estrous. This same pattern also occurred in the olanzapine treated group and in the control group. Individual variations to this pattern did occur. The results suggest that even with confounding values associated with progesterone effects, the average cholesterol value of 186.8 for the compound of the invention is well within the normal beagle dog reference range. As demonstrated in a previous study on a smaller group of animals, there is no rise in cholesterol at a dose of 4 mgs/kg/day in male or female beagle dogs and there is no rise in cholesterol in male beagle dogs at 8 mgs/kg/day relative to placebo for either the 222 compound or olanzapine. These results suggest that the male beagle dog is a better model to assess cholesterol values associated with a possible drug effect since there is no possibility of a progesterone confounding effect in the male. These results further suggest that the female beagle dog is an inappropriate model to predict drug effects on cholesterol metabolism in humans. Olanzapine, in fact, has been associated recently with elevated cholesterol levels and diabetes in humans. The drug, however, is still on the market and has not been pulled or withdrawn for safety reasons.

In addition to total cholesterol values other parameters were measured including blood glucose levels at pretest, week 13 and week 26 of the study. The values for olanzapine were higher than the control values at weeks 13 and 26. The values for the 222 compound were closer to the control values at these same time points. This may suggest that the 222 compound has less of an effect on glucose metabolism relative to control than olanzapine and thus might be less likely to impact diabetes in humans. Control (pretest: 82.2; week 13: 84.8; week 26 77.9 mg/dL); 222 (pretest: 82.5; week 13: 87.9; week 26: 77.6 mg/dL); olanzapine (pretest: 84.1; week 13: 91.9; week 26: 81.6 mg/dL). There is a need, therefore, for a compound that is effective at treating CNS disorders such as schizophrenia but which does not elevate cholesterol in humans or contribute to diabetes.

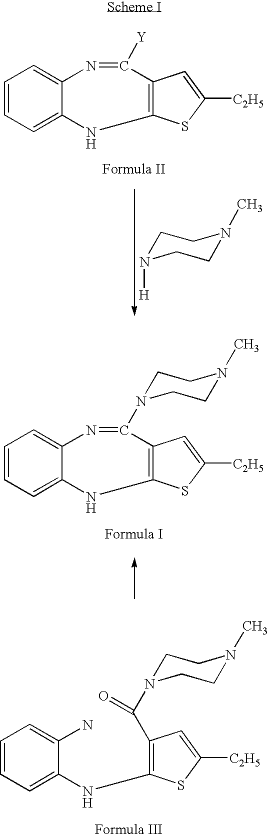

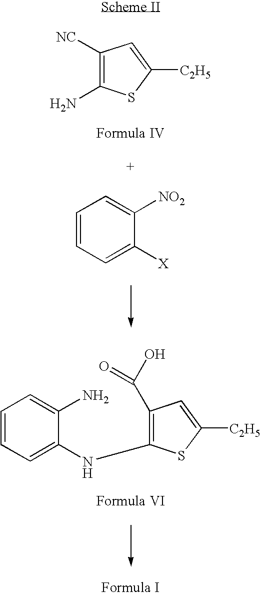

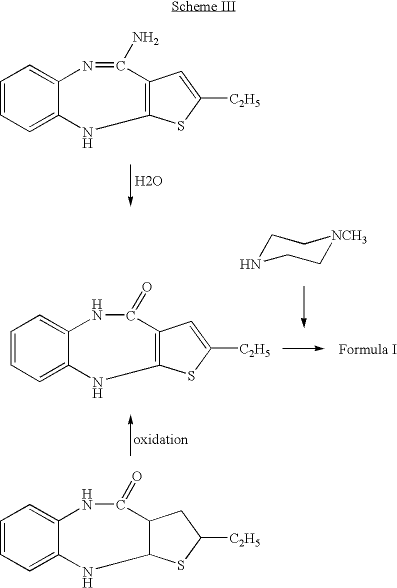

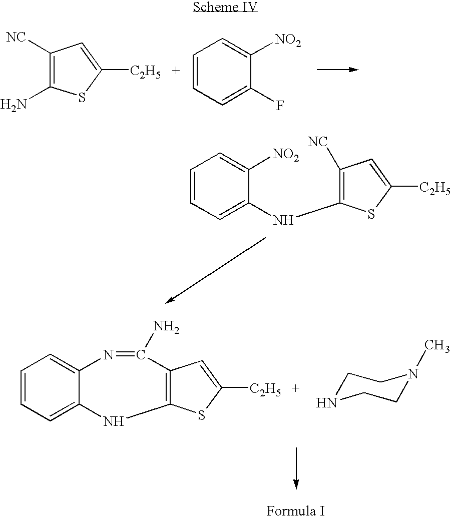

We claim:

1. 2-ethyl-10-(4-methyl-1-piperazinyl)-4H-thieno[2,3-b][1,5]benzodiazepine, or an acid addition salt thereof or prodrug thereof in substantially pure form.

2. The compound according to claim 1 wherein the substantially pure form is crystalline material.

3. The compound according to claim 1 wherein the substantially pure form is an amorphous material.

4. The compound according to claim 1 wherein the compound is a crystalline hydrate or hemihydrate.

5. The compound according to claim 1 wherein the compound is in the form of a polymorph.

6. A compound selected from 2-ethyl-10-(4-methyl-1-piperazinyl)-4H-thieno[2,3-b][1,5]benzodiazepine having a melting point of 203-206 C.

7. A compound selected from 2-ethyl-10-(4-methyl-1-piperazinyl)-4H-thieno[2,3-b][1,5]benzodiazepine having an average particle size as measured by volumetric means of between 1 and 200 micrometers.

8. A pharmaceutical composition comprising a compound according to claim 1 or a pharmaceutically acceptable acid addition salt or prodrug thereof and a pharmaceutically acceptable excipient.

9. A pharmaceutical composition in capsule or tablet form comprising from about 0.5 mg to about 15 mg of the compound of claim 1 together with a pharmaceutically acceptable excipient.

10. A method of treating a patient in need of treatment thereof suffering from or susceptible to psychosis, acute mania or mild anxiety states, which comprises administering an effective amount of 2-ethyl-10-(4-methyl-1-piperazinyl)-4H-thieno[2,3-b][1,5]benzodiazepine, or a pharmaceutically acceptable acid addition salt or prodrug thereof to said patient.

11. A method according to claim 10 wherein the effective amount is from 0.1 to 20 mg per day of 2-ethyl-10-(4-methyl-1-piperazinyl)-4H-thieno[2,3-b][1,5]benzodiazepine, or a pharmaceutically acceptable acid addition salt or prodrug thereof.

12. A method of treating a patient suffering from or susceptible to schizophrenia comprising administering to said patient a pharmaceutically effective amount of 2-ethyl-10-(4-methyl-1-piperazinyl)-4H-thieno[2,3-b][1,5]benzodiazepine.

13. A method according to claim 12 wherein the effective amount is from 0.1 to 20 mg per day of 2-ethyl-10-(4-methyl-1-piperazinyl)-4H-thieno[2,3-b][1,5]benzodiazepine, or a pharmaceutically acceptable acid addition solution salt or prodrug thereof.

14. A pharmaceutical composition comprising a combination of a pharmaceutically effective amount of 2-methyl-10-(4-methyl-1-piperazinyl)-4H-thieno[2,3-b][1,5]benzodiazepine and 2-ethyl-10-(4-methyl-1-piperazinyl)-4H-thieno[2,3-b][1,5]benzodiazepine, together with pharmaceutically acceptable excipients.

15. A method of treating a patient having or susceptible acute mania comprising administering a pharmaceutically effective amount of 2-ethyl-10-(4-methyl-1-piperazinyl)-4H-thieno[2,3-b][1,5]benzodiazepine or a salt or prodrug thereof.

16. A method of treating a patient having mild anxiety states comprising administering a pharmaceutically effective amount of 2-ethyl-10-(4-methyl-1-piperazinyl)-4H-thieno[2,3-b][1,5]benzodiazepine or a salt or prodrug thereof.

17. A pharmaceutical composition in capsule or tablet form comprising from 0.1 to 20 mg of the compound of claim 1 together with a pharmaceutically acceptable diluent or carrier therefor.

18. A method of treating a CNS disorder or condition comprising administering a pharmaceutically effective amount of the compound 2-ethyl-10-(4-methyl-1-piperazinyl)-4H-thieno[2,3-b][1,5]benzodiazepine or a salt or prodrug thereof wherein, relative to the compound 2-methyl-10-(4-methyl-1-piperazinyl)-4H-thieno[2,3-b][1,5]benzodiazepine, there is no statistically significant elevation in cholesterol levels in patients treated with said compound.

19. A pharmaceutical composition in liquid form wherein the composition comprises a compound selected from 2-ethyl-10-(4-methyl-1-piperazinyl)-4H-thieno[2,3-b][1,5]benzodiazepine or salt or prodrug thereof and a pharmaceutically acceptable diluent.

20. A sustained release delivery system comprising a compound selected from 2-ethyl- or 2-methyl-10-(4-methyl-1-piperazinyl)-4H-thieno[2,3-b][1,5]benzodiazepine and sustained release excipients.

21. An immediate release delivery system comprising a compound selected from 2-ethyl-10-(4-methyl-1-piperazinyl)-4H-thieno[2,3-b][1,5]benzodiazepine or salt or prodrug thereof and immediate release excipients.

22. A quick dissolving formulation comprising 2-ethyl-10-(4-methyl-1-piperazinyl)-4H-thieno[2,3-b][1,5]benzodiazepine or salt or prodrug thereof and quick dissolving excipients.

23. A combination comprising a compound selected from olanzapine or the 2-ethyl homolog thereof together with an HMG Co-A reductase inhibitor as a second active ingredient.

24. The combination according to claim 23 in the form of an oral dosage form having the two active ingredients in said dosage form.

25. The oral dosage form according to claim 24 comprising a tablet or capsule.

26. A method of treating a patient having a CNS disorder comprising administering to said patient a combination of a therapeutically effective amount of olanzapine or the 2-ethyl homolog thereof and an HMG CoA reductase inhibitor and wherein the HMG CoA reductase inhibitor is present in a pharmaceutically effective amount to minimize or mitigate any cholesterol raising effect of olanzapine or the 2-ethyl homolog thereof.

27. The method according to claim 26 wherein the CNS disorder is schizophrenia.

28. The method according to claim 26 wherein the combination is in the form of an oral dosage form having the two active ingredients in said dosage form.

29. The method according to claim 26 wherein the CNS disorder is bipolar disorder.

30. The method according to claim 26 wherein the HMG CoA reductase inhibitor is selected from lovastatin or simvastatin or pravastatin.

31. The method according to claim 30 wherein simvastatin or lovastatin is present in a dosage range of 1-20 mgs/day.

32. The method according to claim 26 wherein the dosage form contains an additional active ingredient selected from an antidepressant.

33. A pharmaceutical composition comprising 2-ethyl-10-(4-methyl-1-piperazinyl)-4H-thieno[2,3-b][1,5]benzodiazepine or salt or prodrug thereof and a pharmaceutically acceptable excipient or diluent wherein said composition, when administered to female beagle dogs over a twenty six week period, resulted in a total average cholesterol value of between 140 and 240 mg/dL.

34. A pharmaceutical composition comprising 2-ethyl-10-(4-methyl-1-piperazinyl)-4H-thieno[2,3-b][1,5]benzodiazepine or salt or prodrug thereof and a pharmaceutically acceptable excipient or diluent wherein said composition, when administered to female beagle dogs over a twenty six week period, resulted in a total average cholesterol value of between 150 and 220 mg/Dl.

35. A pharmaceutical composition comprising 2-ethyl-10-(4-methyl-1-piperazinyl)-4H-thieno[2,3-b][1,5]benzodiazepine or salt or prodrug thereof and a pharmaceutically acceptable excipient or diluent wherein said composition, when administered to female beagle dogs over a twenty six week period, resulted in a total average cholesterol value of between 180 and 210 mg/Dl.

36. A method of treating a human patient in need of treatment thereof for a CNS disorder or condition selected from a psychotic disorder including schizophrenia or bipolar mania comprising administering a pharmaceutically effective amount of the compound 2-ethyl-10-(4-methyl-1-piperazinyl)-4H-thieno[2,3-b][1,5]benzodiazepine or salt or prodrug thereof to said patient wherein there is no significant elevation in said patients cholesterol levels.

37. A method of treating a human patient in need of treatment thereof for a CNS disorder or condition selected from a psychotic disorder including schizophrenia or bipolar mania comprising administering a pharmaceutically effective amount of the compound 2-ethyl-10-(4-methyl-1-piperazinyl)-4H-thieno[2,3-b][1,5]benzodiazepine or salt or prodrug thereof to said patient wherein there is an elevation in said patients of cholesterol levels.

38. A method of treating a human patient in need of treatment thereof for a CNS disorder or condition selected from a psychotic disorder including schizophrenia or bipolar mania comprising administering a pharmaceutically effective amount of the compound 2-ethyl-10-(4-methyl-1-piperazinyl)-4H-thieno[2,3-b][1,5]benzodiazepine or salt or prodrug thereof to said patient wherein the patient's total cholesterol level is between 120-220 mg/Dl.

39. The method according to claim 37 wherein said patient is concomitantly treated with an HMG CoA reductase inihbitor.

40. A method of treating a human patient in need of treatment thereof for a CNS disorder or condition selected from a psychotic disorder including schizophrenia or bipolar mania comprising administering a pharmaceutically effective amount of the compound 2-ethyl-10-(4-methyl-1-piperazinyl)-4H-thieno[2,3-b][1,5]benzodiazepine or salt or prodrug thereof to said patient wherein the patient has minimal weight gain attributable to administration of said compound.

41. A method of treating a human patient in need of treatment thereof for a CNS disorder or condition selected from a psychotic disorder including schizophrenia or bipolar mania comprising administering a pharmaceutically effective amount of the compound 2-ethyl-10-(4-methyl-1-piperazinyl)-4H-thieno[2,3-b][1,5]benzodiazepine or salt or prodrug thereof to said patient wherein the patient suffers minimal dyskenisia.

42. A compound having an X-ray diffraction pattern with d values and relative intensities of 6.56 (13.9%); 6.21 (39.5%); 5.31 (11.7%); 5.01 (29%); 4.71 (15.2%); 4.42 (38.7%); 4.15 (100%); 3.28 (18%) and 3.11 (10.3%).

43. The compound according to claim 42 having a melting point of 203-206 degrees C.

44. A pharmaceutical composition comprising a compound having an X-ray pattern according to claim 42 and a pharmaceutically acceptable excipient.

45. A method of treating a CNS disorder comprising administering to a patient in need of treatment thereof a compound having an X-ray diffraction pattern according to claim 42.
